(19) United States
(12) Patent Application Publication
(10) Pub. No.: US 2014/0005714 A1
(43) Pub. Date: Jan. 2, 2014

(54) MULTILAYERED EXPANDABLE BRAIDED DEVICES AND METHODS OF USE

(71) Applicant: INCEPTUS MEDICAL, INC., (US)

(72) Inventors: Richard Quick, Mission Viejo, CA (US); Paul Lubock, Monarch Beach, CA (US); Robert Rosenbluth, Laguna Niguel, CA (US); Brian J. Cox, Laguna Niguel, CA (US)

(73) Assignee: INCEPTUS MEDICAL, INC., Aliso Viejo, CA (US)

(21) Appl. No.: 13/844,232

(22) Filed: Mar. 15, 2013

Related U.S. Application Data
(63) Continuation-in-part of application No. PCT/US13/20381, filed on Jan. 4, 2013.
Application No. PCT/US13/20381 is a continuation-in-part of application No. PCT/US12/51502, filed on Aug. 17, 2012.
(60) Provisional application No. 61/583,993, filed on Jan. 6, 2012.
(60) Provisional application No. 61/636,392, filed on Apr. 20, 2012.

(30) Foreign Application Priority Data
Aug. 17, 2012 (WO) PCTUS1251502
Jan. 4, 2013 (WO) PCTUS1320381

(51) Int. Cl. A61B 17/12

(52) U.S. Cl. CPC A61B 17/12122
USPC 606/200

(57) ABSTRACT

Devices and methods for occluding blood flow into a body cavity are disclosed herein. An occlusion device can include an expandable lattice structure having an occlusive braid and a structural braid. In some embodiments, the structural braid is enveloped by the occlusive braid, and the occlusive braid is configured to contact and seal with tissue. The structural braid can have a first pick angle and the occlusive braid can have a second pick angle that is greater than the first pick angle. In some embodiments, the occlusion device is configured to occlude the left atrial appendage.

### CROSS-REFERENCE TO RELATED APPLICATIONS

The present application is a continuation-in-part of International Patent Application No. PCT/US2013/20381, entitled “EXPANDABLE OCCLUSION DEVICES AND METHODS OF USE,” which claims priority to U.S. Provisional Application No. 61/583,993, filed Jan. 6, 2012, entitled “DEVICES AND METHOD FOR OCCLUSION OF THE LEFT ATRIAL APPENDAGE,” U.S. Provisional Application No. 61/636,392, filed Apr. 20, 2012, entitled “DEVICES AND METHODS FOR VASCULAR OCCLUSION,” and PCT Application No. PCT/US12/51502, filed Aug. 17, 2012, entitled “EXPANDABLE OCCLUSION DEVICES AND METHODS,” the full disclosures of which are incorporated herein by reference.

### TECHNICAL FIELD

The present technology relates generally to cardiovascular devices, implant delivery systems, and methods of using cardiovascular devices and delivery systems to treat structural and functional defects in the heart and circulatory system. More specifically, the present technology is directed to the occlusion of undesirable blood flow into cavities such as the left atrial appendage.

### BACKGROUND

FIGS. 1 and 2 show a heart (“H”) and a left atrial appendage (“LAA”). The LAA is a muscular pouch or cavity connected to the lateral wall of the left atrium (“LA”) of the heart H between the mitral valve and the roots of the left superior and left inferior pulmonary veins (“LSPV” and “LIPV,” respectively). Although the exact function of the LAA is not known, during normal left atrial filling the LAA also fills and blood is expelled with the contraction of the left atrium LA. In some disease states, particularly a condition known as atrial fibrillation that is estimated to afflict over 5 million people worldwide, the contraction of the LAA may be inhibited or inconsistent and pooling of blood in the LAA may occur. The pooled blood may clot and subsequently embolize into the arterial circulation potentially leading to embolic stroke of the brain, heart or other vital organs.

To reduce the incidence of stroke, patients with atrial fibrillation are typically placed on lifelong anticoagulation and/or antiplatelet medications. These medications have several potential drawbacks including risk of bleeding, adverse side effects, inability of the patient to titrate the appropriate dose, inconvenience, high cost, low compliance and others. In practice, the estimated number of atrial fibrillation patients adequately receiving medication is less than 50%. Other treatment options include thoracoscopic surgical removal and ligation of the LAA, but these procedures also have several drawbacks including exclusion of high surgical risk candidates, high morbidity, mortality risk, infection, and others.

Less invasive approaches to LAA occlusion have been developed in recent years, such as transcatheter LAA occlusion. Transcatheter occlusion devices are generally placed percutaneously with a catheter positioned through the femoral vein to the right heart and then transeptally to the left atrium and into the LAA under fluoroscopic and/or ultrasound guidance. These devices, however, have drawbacks such as insufficient sealing at the ostium, inadequate fixation of the device, poor hemodynamic design leading to excessive thrombo-emboli in the atrium, and other drawbacks described in more detail below. Accordingly, there is a need for devices and methods that address one or more of these deficiencies.

### BRIEF DESCRIPTION OF THE DRAWINGS

The accompanying drawings, which are incorporated herein and constitute part of this specification, illustrate embodiments of the present technology, and, together with the general description given above and the detailed description given below, serve to explain the features of the present technology.

FIG. 1 is a posterior view of a heart.

FIG. 2 is a posteroinferior view of a heart.

FIG. 3 a side perspective view an expandable occlusion device in a deployed state (e.g., expanded configuration) for placement in a body cavity in accordance with an embodiment of the present technology.

FIG. 4A is a side view of an occlusion device for placement within the cavity of the body in accordance with an embodiment of the present technology.

FIG. 4B is a cross-sectional side view of an expandable occlusion device having an occlusive braid and a structural braid configured in accordance with an embodiment of the present technology.

FIG. 4C is an enlarged view of a proximal hub of FIG. 4B, in accordance with embodiments of the present technology.

FIG. 4D is an enlarged view of an outer distal hub of FIG. 4B, in accordance with embodiments of the present technology.

FIG. 4E is a side view of an occlusion device comprising a single layer occlusive braid, in accordance with embodiments of the present technology.

FIG. 5A is a perspective view of an expanded occlusion device having retention members in accordance with an embodiment of the present technology.

FIG. 5B is an enlarged cross-sectional view of a section of FIG. 5A in accordance with an embodiment of the present technology.

FIGS. 5C-5K show different embodiments of retention members in accordance with the present technology.

FIG. 5L is a perspective view of an expanded occlusion device having retention members in accordance with an embodiment of the present technology.

FIG. 5M is a perspective cross-sectional view of an expanded occlusion device having an outer anchoring lattice configured in accordance with an embodiment of the present technology.

FIG. 6A is a schematic cross-sectional view of one embodiment of a delivery system is configured in accordance with an embodiment of the present technology.

FIG. 6B is an enlarged cross-sectional side view of select components at a distal region of an occlusion device delivery system in accordance with an embodiment of the present technology.

FIGS. 7A-7C show a typical antegrade approach to the left atrial appendage of the heart.

FIG. 7D is a side perspective view of a guidewire and delivery catheter positioned at the left atrial appendage in accordance with an embodiment of the present technology.

FIG. 7E is a side perspective view of a partially expanded occlusion device during deployment at the left atrial appendage in accordance with an embodiment of the present technology.

FIG. 7F is a side perspective view of an expandable occlusion device in a deployed state (e.g., expanded configuration) positioned at the left atrial appendage in accordance with an embodiment of the present technology.

FIG. 8A is a schematic side view of one embodiment of a delivery system having a balloon positioning member in accordance with an embodiment of the present technology.

FIG. 8B is a schematic side view of one embodiment of a delivery system having an expandable mesh positioning member in accordance with an embodiment of the present technology.

FIG. 8C is a schematic side view of one embodiment of a delivery system having a Malecot positioning member in accordance with an embodiment of the present technology.

FIG. 8D is a schematic side view of one embodiment of a delivery system having a mechanical positioner positioning member in accordance with an embodiment of the present technology.

FIG. 9A is a schematic cross-sectional side view of one embodiment of an occlusion device delivery system having a retention member actuation mechanism in accordance with an embodiment of the present technology.

FIG. 9B is a schematic cross-sectional side view of one embodiment of an occlusion device delivery system having a retention member actuation mechanism in accordance with an embodiment of the present technology.

FIG. 10A is a schematic side view of one embodiment of an occlusion device retention member actuation in accordance with an embodiment of the present technology.

FIG. 10B is a schematic side view of one embodiment of an occlusion device retention member actuation in accordance with an embodiment of the present technology.

FIG. 11A is an enlarged view of a self-expanding braid with interwoven large and small strands configured in accordance with an embodiment of the present technology.

FIG. 11B is a side view of a mandrel and a braided mesh formed over the mandrel configured in accordance with an embodiment of the present technology.

FIG. 12A is a schematic side view of an occlusion device having a proximal section and a distal section in accordance with an embodiment of the present technology.

FIG. 12B is a schematic side view of an occlusion device having a proximal section with a flange in accordance with an embodiment of the present technology.

FIG. 12C is a schematic side view of an occlusion device having a proximal section, a middle section, and a distal section in accordance with an embodiment of the present technology.

FIG. 12D is a schematic side view of an occlusion device having annular sections in accordance with an embodiment of the present technology.

FIG. 12E is a schematic side view of an occlusion device having a proximal section and a distal section coupled by a spring in accordance with an embodiment of the present technology.

FIG. 12F is a schematic side view of an occlusion device having a mechanically coupled proximal section and distal section in accordance with an embodiment of the present technology.

FIG. 13A is a schematic cross-sectional side view of an occlusion device having nested sections, in accordance with an embodiment of the present technology.

FIG. 13B is a schematic side view of the occlusion device of FIG. 13A when stretched, in accordance with an embodiment of the present technology.

FIG. 14 is a side cross-sectional view of a portion of an occlusion device in accordance with an embodiment of the present technology.

FIGS. 15A and 15B are diagrams showing the relationship between the pick angle and lengths of braids used in multi-braid devices.

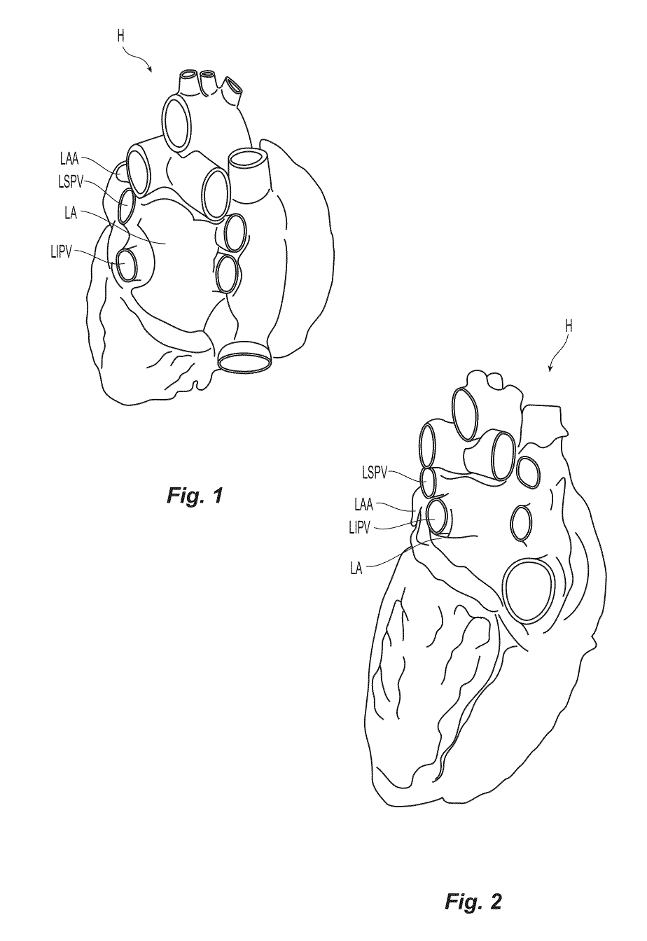

### DETAILED DESCRIPTION

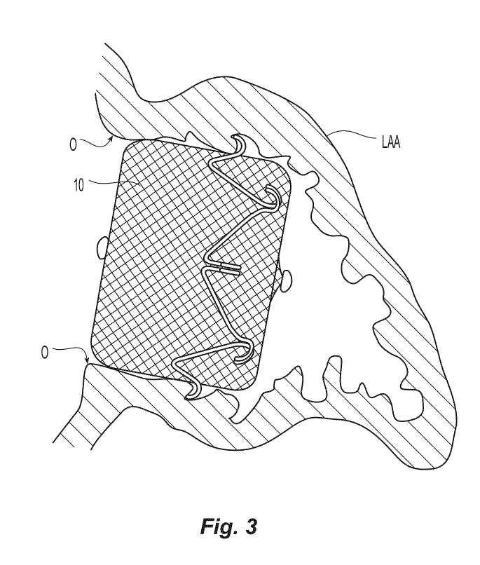

Specific details of several embodiments of the technology are described below with reference to FIGS. 3-14. Although many of the embodiments are described below with respect to devices, systems, and methods for occlusion of the left atrial appendage, other applications and other embodiments in addition to those described herein are within the scope of the technology. Additionally, several other embodiments of the technology can have different configurations, components, or procedures than those described herein. A person of ordinary skill in the art, therefore, will accordingly understand that the technology can have other embodiments with additional elements, or the technology can have other embodiments without several of the features shown and described below with reference to FIGS. 3-14.

With regard to the terms “distal” and “proximal” within this description, unless otherwise specified, the terms can reference a relative position of the portions of an occlusion device and/or an associated delivery device with reference to an operator and/or a location in the vasculature. For example, proximal can refer to a position closer to the operator of the device or an incision into the vasculature, and distal can refer to a position that is more distant from the operator of the device or further from the incision along the vasculature.

For ease of reference, throughout this disclosure identical reference numbers are used to identify similar or analogous components or features, but the use of the same reference number does not imply that the parts should be construed to be identical. Indeed, in many examples described herein, identically numbered parts of individual embodiments are distinct in structure and/or function. The headings provided herein are for convenience only.

### 1. Selected Embodiments of Occlusion Devices

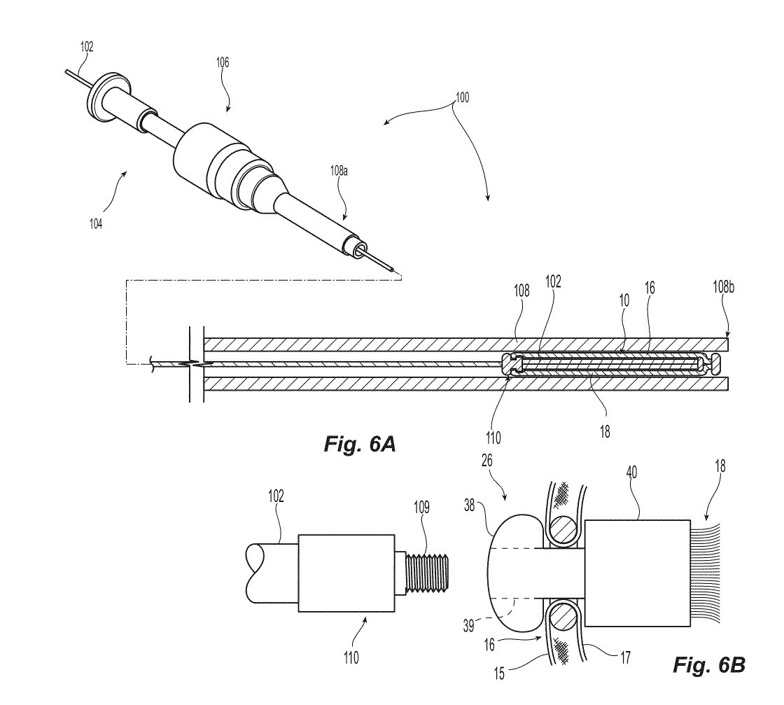

Introductory examples of occlusion devices, systems and associated methods in accordance with embodiments of the present technology are described in this section with reference to FIGS. 3-5M. It will be appreciated that specific elements, substructures, advantages, uses, and/or other features of the embodiments described with reference to FIGS. 3-5M can be suitably interchanged, substituted or otherwise configured with one another and/or with the embodiments described with reference to FIGS. 6A-14 in accordance with additional embodiments of the present technology. Furthermore, suitable elements of the embodiments described with reference to FIGS. 3-5M can be used as standalone and/or self-contained devices.

Several embodiments of systems, devices and methods for occluding a body cavity described below are particularly well suited for occluding the LAA of the heart. FIG. 3 shows an embodiment of an occlusion device 10 deployed (i.e., expanded configuration) within the LAA ostium (“O”). As shown, the left atrium LA is proximal the ostium O of the LAA, and the ostium O of the LAA is proximal to the cavity of the LAA. The cavity of the LAA is accordingly distal to the left atrium LA. The occlusion device 10 can include an expandable lattice structure having a proximal region configured to be positioned at or near the ostium of the LAA, a distal region configured to extend into an interior portion of the LAA, and a contact region between the proximal and distal portions. In several embodiments, the expandable lattice structure includes an occlusive braid configured to contact and seal with tissue of the LAA and a structural braid enveloped by the occlusive braid. The structural braid can be coupled to the occlusive braid at a proximal hub located at the proximal region of the lattice structure. The structural braid is configured to drive the occlusive braid radially outward. The occlusive braid can have an atrial face at the proximal portion facing the left atrium LA, and the atrial face can have a low-profile contour that mitigates thrombus formation at the atrial face.

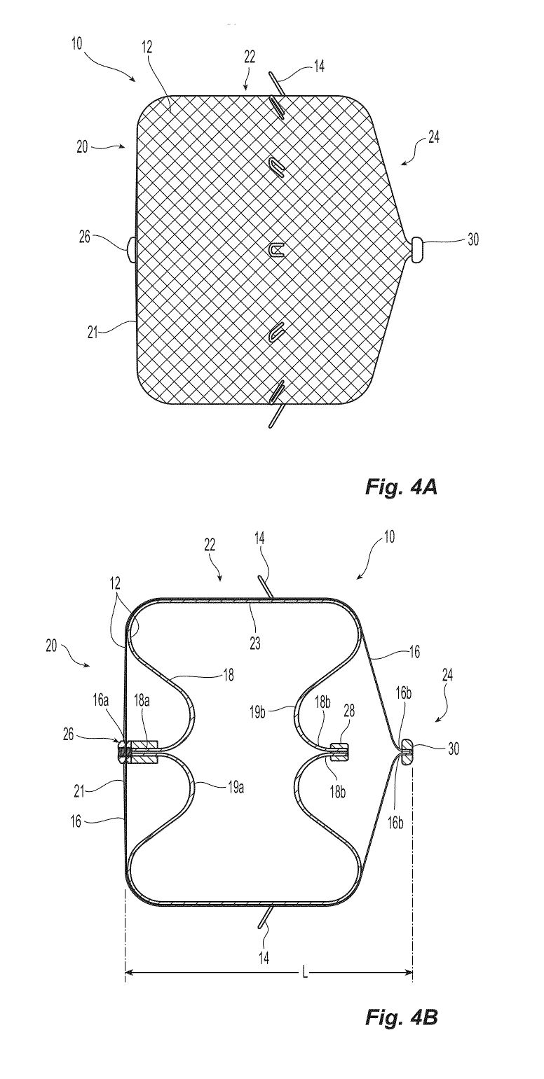

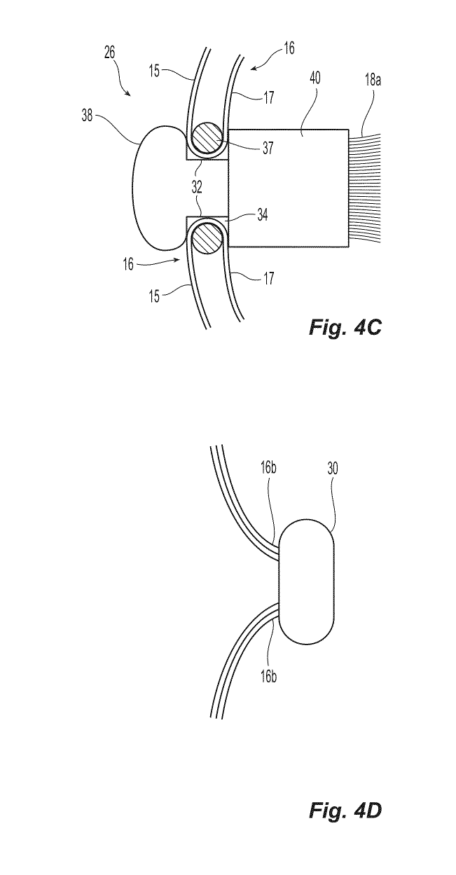

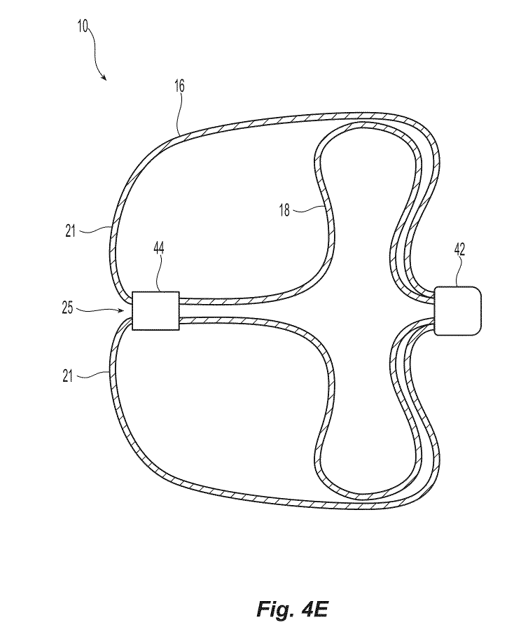

FIGS. 4A-4D show one embodiment of an occlusion device 10 in an unrestricted expanded configuration. As shown in the side view of FIG. 4A, the occlusion device 10 includes a flexible, self-expanding lattice structure 12 and one or more retention members 14 coupled to and/or integrated with the lattice structure 12. The lattice structure 12 can be generally cylindrical, as shown in FIG. 4A. In other embodiments, the lattice structure 12 can have a shape that is generally spherical, ellipsoidal, oval, barrel-like, conical, frustrum-shaped, or any other suitable shape. The lattice structure 12 can have a proximal region 20 having a low-profile atrial face 21, a distal region 24, and a contact region 22 in between. As shown in FIG. 4A, in some embodiments the atrial face 21 can be planar or substantially planar with a slight proximal and/or distal bow, the contact region 22 can be generally cylindrical, and the distal region 24 can be tapered. The contact region 22 can provide a sufficient outward radial force to deform the LAA to a certain extent while also being sufficiently flexible to conform to the LAA such that the contact region becomes at least substantially sealed to the LAA tissue.

The lattice structure 12 can include one or more layers, and each layer can comprise an expandable lattice and/or a braided mesh of filaments (e.g., wires, threads, sutures, fibers, etc.). For example, as shown in the cross-sectional side view of FIG. 4B, the lattice structure 12 can include an occlusive braid 16 and a structural braid 18 arranged so that the occlusive braid 16 envelops the structural braid 18. Both the occlusive braid 16 and the structural braid 18 have proximal ends 16a and 18a, respectively, secured to a proximal hub 26. The outer occlusive braid 16 has distal ends 16b secured to an outer distal hub 30 and the inner structural braid 18 has distal ends 18b secured to an inner distal hub 28. The inner distal hub 28 moves independently of the outer distal hub 30 such that the occlusive and structural braids 16 and 18 can have different lengths without causing one of the braids to bunch upon collapse for delivery because the braids can move relative to each other to accommodate compression into a contracted state.

As illustrated in FIG. 4C, a substantial portion of the proximal hub 26 is encapsulated by the occlusive braid 16. Because of this, only a small portion of the hub protrudes from the atrial face 21 such that the proximal hub 26 only has a slight or negligible effect on the profile of the atrial face 21. For example, in some embodiments, the proximal hub 26 increases the profile of the atrial face 21 by less than 2 mm in the proximal direction, or in some embodiments, by less than 1 mm. Accordingly, the atrial face 21 can include a proximal hub 26 and still maintain a low-profile contour. A low-profile atrial face is important since thrombi can potentially form at or along any surface of the device that is exposed to blood flow. Many existing devices have structures at a proximal region of the device which protrude into the left atrium. These protrusions increase the surface area of the device at a high blood flow region (i.e., at or near an atrial chamber of the heart), thus increasing the likelihood of thrombus formation on the device. Similarly, grooves and/or pockets at a proximal region of the device present the same risk. The substantially planar atrial face 21 of the proximal region 20 mitigates this risk, as does the porous nature of the lattice structure 12. It is believed that clots formed on smooth surfaces are more likely to embolize into the bloodstream than clots formed on a porous surface. The atrial face 21 of the present invention comprises a plurality of interstices (i.e., the lattice structure) in which a thrombus or portion of a thrombus can get stuck, thus decreasing the likelihood of embolization of that thrombus.

Referring to FIG. 4D, the outer distal hub 30 can have an atraumatic shape. For example, the distal hub 30 can have a cross-sectional shape such as a sphere, an oval, an ellipse, a hemisphere with a rounded edge, a “mushroom-top” shape (see FIG. 4D), and others. The outer distal hub 30 secures the distal ends 16a of the occlusive braid and serves as an extension of the occlusion device 10 that can easily be snared should the device embolize into the left atrium during and/or after placement. Several existing devices have structures and/or extensions along the length of the device or at a distal region which can cause unnecessary trauma to the LAA during and/or after deployment.

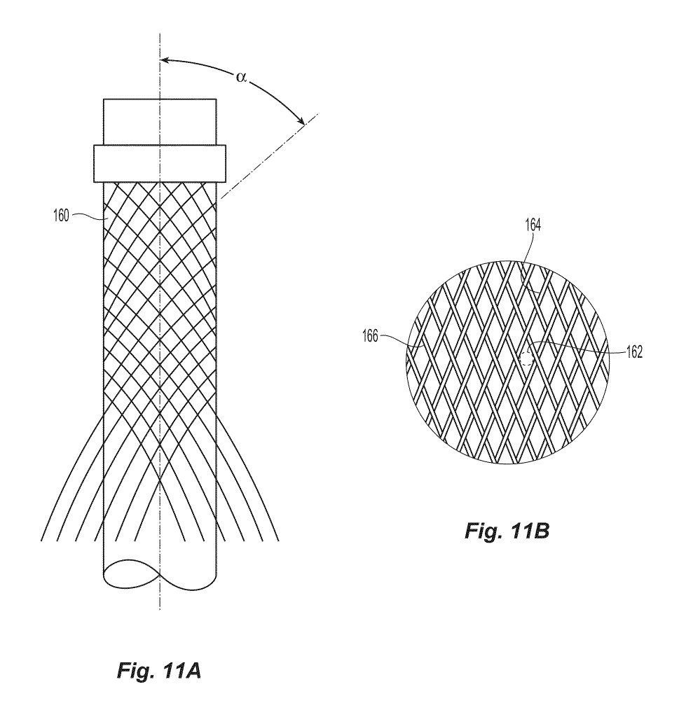

Referring to FIGS. 4B-4C, the outer occlusive braid 16 can have an external layer 15 and an internal layer 17 created by everting the occlusive braid 16 around an edge 32 at each of its proximal ends 16a. In other embodiments, the occlusive braid 16 can have more or less than two layers (as discussed below with reference to FIG. 4E). As shown in the enlarged view of the proximal hub 26 in FIG. 4C, the proximal hub 26 can have an inner portion 40 within the occlusive braid 16, a cap 38 coupled to the inner portion 40, and a groove 34 between the cap 38 and the inner portion 40. The edges 32 of the occlusive braid 16 can be received in the groove 34. For example, a clamp ring 37 can urge the edges 32 inwardly to secure the occlusive braid 16 to the proximal hub 26. The proximal ends 18a of the structural braid 18 can be secured to the inner portion 40 of the proximal hub 26. The characteristics of the occlusive braid 16 can remain constant as the braided mesh continues around the everted portion 32, or it can be formed with two or more braiding techniques so that the braiding on the inside for the internal layer 17 is different than the braiding on the outside for the external layer 15. Likewise, the braiding can change to provide differing braid angles and/or pore sizes between layers and/or along the length of the occlusive braid 16, as discussed in greater detail below with reference to FIGS. 11A-11B. For example, the maximum pore size of any pore on the atrial face 21 of the occlusive braid 16 can be less than 0.6 mm. In some embodiments, the maximum pore size of any pore on the atrial face 21 is less than 0.5 mm. Referring to FIG. 4D, the distal ends 16b of the occlusive braid 16 can be secured to the outer distal hub 30 by welding.

The mesh of the occlusive braid 16 can be configured to at least substantially, if not totally, occlude blood flow into the LAA and provide a biocompatible scaffold to promote new tissue ingrowth. The occlusive braid 16 can be made from a braided mesh of metal filaments, including nickel-titanium alloys (e.g. Nitinol), platinum, cobalt-chrome alloys, Elgiloy, stainless steel, tungsten or titanium. In some embodiments, it is desirable that the occlusive braid 16 be constructed solely from metallic materials free of any polymer materials. It is believed that the exclusion of polymer materials in some embodiments may decrease the likelihood of thrombus formation on device surfaces. It is further believed that the exclusion of polymer materials in the occlusive and/or structural braids and the sole use of metallic components can provide an occlusion device with a thinner profile that can be delivered with a small catheter as compared to devices having polymeric components. For example, the delivery catheter can be about 5F to 24F, and in some embodiments, 6F to 15F. In some embodiments, the delivery catheter can be about 8F-12F.

Some existing devices include a self-expanding frame at least partially covered at an atrial region by a permeable polymer (i.e., polyester) fabric. If the device is improperly sized and does not fully expand, the polymer fabric may loosen and/or “buckle” between the struts of the frame, much like fabric of an umbrella that has folds when not fully expanded. This can cause leakage around the device as well as create grooves for potential thrombus formation, as discussed above. Furthermore, many existing devices comprise a substantially circular cross-section while the LAA ostium generally has an oval-shaped cross-section. These devices rely on the LAA to adapt and conform to the device, which can also cause inadequate sealing at the LAA ostium. Although the occlusive braid 16 and structural braid 18 may be in contact along a portion of the lattice structure, the braids 16 and 18 are coupled only at a proximal region, allowing for space and free movement of the occlusive braid 16 along the length L of the lattice structure 12. The mesh of the occlusive braid 16 can be configured to have a pore size, filament diameter, weave density, and/or shape to create a highly flexible outer layer that can conform and/or generally comply to the surface of the LAA. For example, the occlusive braid 16 can have pore sizes (described below with reference to FIG. 11A) in the range of about 0.025 mm to 2.0 mm. In some embodiments, the occlusive braid 16 can have pores size in the range of 0.025 mm to 0.300 mm, outside the range of existing devices.

The structural braid 18 can comprise the innermost layer of the lattice structure 12 and stabilizes and shapes the occlusive braid 16 and/or other layers of the lattice structure 12. When expanded, the structural braid 18 can include a generally cylindrical contact portion 23 that extends proximally along a proximal folded portion 19a and extends distally along a distal folded portion 19b. When expanded, the contact portion 23 drives the occlusive braid 16 radially outward to contact the LAA wall and/or trabeculae. The radial force exerted by the structural braid 18 can be substantially uniformly radial and is generally sufficient to inhibit movement, dislodgement and potential embolization of the occlusion device 10. Depending on the sizing of the lattice structure 12 and/or occlusion device 10, the LAA wall and/or trabeculae may exert a radially compressive force on the contact portion 23 (i.e., through the occlusive braid 16). The compressive force is then distributed proximally and distally along the length L of the structural braid 18 to the folded portions 19a and 19b which can fold/bend/buckle in response. In some embodiments, the structural braid 18 has an undulating proximal and/or distal portion. Accordingly, compression of the structural braid 18 can have only a slight or negligible impact on the length L of the device. In other words, a decrease in the structural braid 18 diameter has approximately no effect on the length of the contact portion 23 or slightly shortens the length of the contact portion 23. Likewise, the longitudinal distance between the proximal hub 26 and the inner distal hub 28 remains approximately the same or slightly decreases. For example, a 20% change in the diameter of the structural braid 18 can change the length of the contact portion 23 by less than 5%, and in some embodiments, by less than 1%. In some embodiments, a 50% change in the diameter of the structural braid 18 changes the length of the contact portion 23 by less than 5%. This feature is often desirable in LAA occlusion devices since the LAA cavity is relatively short and may vary from patient to patient. Many existing devices lengthen upon implantation due to radially compressive forces at the ostium or LAA wall which can affect proper positioning of the device.

Although the embodiment of an occlusion device 10 shown in FIGS. 4A-4B shows a planar or substantially planar atrial face 21, in some embodiments the low-profile atrial face 21 may have an arcuate, conical, and/or undulating contour. For example, FIG. 4E shows an embodiment of a frustum-shaped occlusion device 10 having an undulating atrial face 21. The lattice structure can be formed with a one-layer occlusive braid 16 having proximal ends coupled to a proximal region of a proximal hub 44 while the proximal ends of the structural braid 18 can be coupled to a distal region of the proximal hub 44. Accordingly, the proximal hub 44 is almost entirely encapsulated by a proximal region of the occlusive braid 16. As a result, the low-profile atrial face 21 is generally flat with a slight depression 25 along a longitudinal axis of the device 10. The slight depression 25 does not substantially disrupt the hemodynamics in the left atrium nor have a significant effect on the profile of the atrial face 21. For example, in some embodiments, such bellows and/or undulations increase and/or decrease the profile of the atrial face 21 by less than 2 mm in the proximal direction. In other embodiments, such bellows and/or undulations increase and/or decrease the profile of the atrial face 21 by less than 1 mm in the proximal direction. As shown in FIG. 4E, the distal ends of the occlusive braid 16 and the distal ends of the structural braid 18 can be coupled to a proximal region of a distal hub 42. In some embodiments, the lattice structure can comprise a single layer including both occlusive and structural properties.

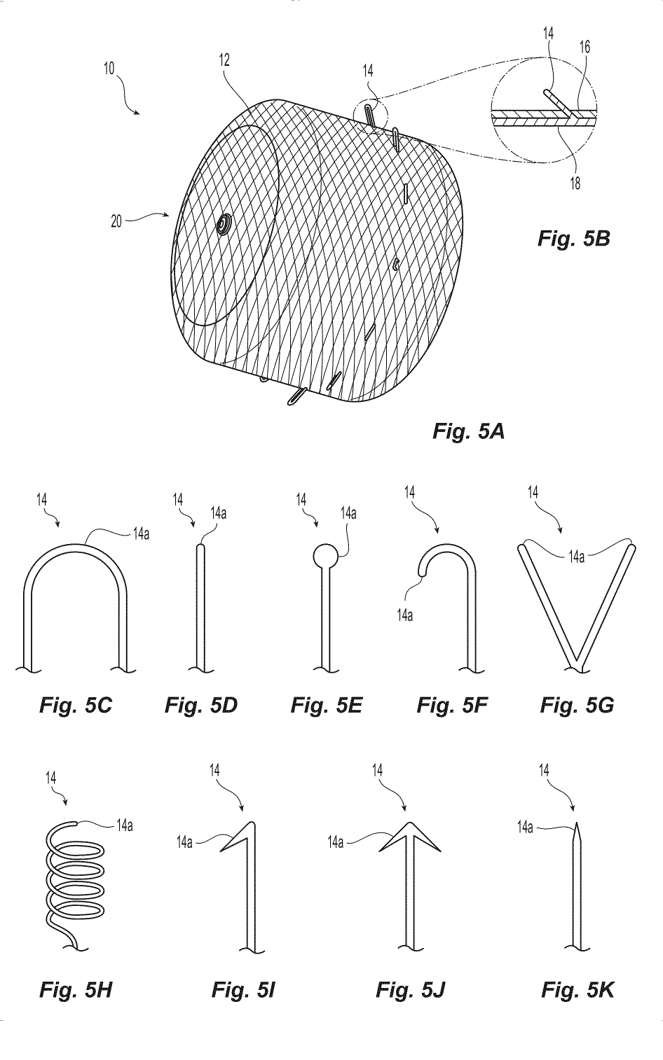

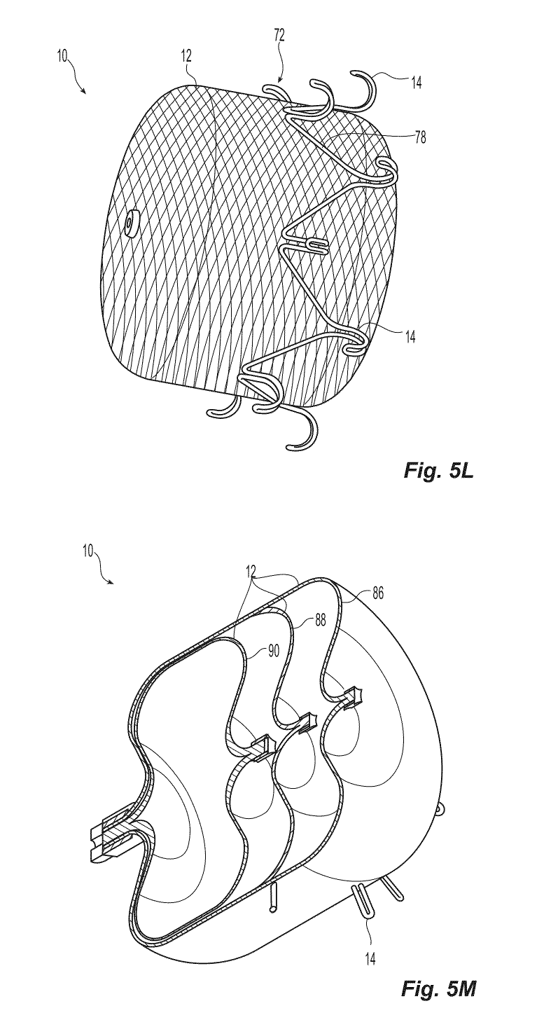

In some embodiments of the device, the occlusion device 10 may incorporate one or more atraumatic and/or non-tissue-penetrating retention members 14 to further secure the occlusion device 10 to at least a portion of the inner wall of the LAA. FIGS. 5A-5B show one embodiment of an occlusion device 10 having retention members 14 arranged around the circumference of the device 10. As shown in the enlarged view of FIG. 5B, the retention members 14 may be contiguous or integrated with the structural braid 18 and pulled through the outer occlusive braid 16 to a point beyond the exterior of the device 10. Retention members 14 may be angled towards a proximal region 20 of the device 10 but are flexible enough to bend and/or conform in response to the LAA anatomy.

Many existing devices fail to fully seal and/or fixate to the LAA anatomy, especially the portions of the LAA wall having trabeculae, and thus fail to adequately secure the occlusion device in the LAA. To combat this issue, some existing devices include members with traumatic or tissue-penetrating shapes and/or ends coupled to the occlusion device. Such traumatic members may perforate the LAA walls causing pericardial effusion and even cardiac tamponade. To avoid these serious conditions, the retention members 14 of the present technology can have an atraumatic shape and are configured to capture and/or interface with the trabeculae without puncturing the trabeculae or the LAA walls. For example, FIGS. 5C-5G and show embodiments of retention members 14 having atraumatic shapes and/or ends 14a. The retention member 14 can be a u-shaped loop (FIG. 5C), a straight wire (FIG. 5D), a straight or bent wire with a spherical end 14a (FIG. 5E), a bent wire (FIG. 5F), a diverging wire have one or more ends 14a (FIG. 5G), and other suitable shapes and/or configurations.

In some embodiments, the occlusion device may additionally or alternatively include traumatic and/or tissue-penetrating retention members which can include at least one fixation member such as a tine, barb, hook (FIG. 5I), pin (FIG. 5K), anchor (FIG. 5J) and others along at least a portion of the retention member 14 and/or at the end 14a of the retention member 14. In some embodiments, the length of the fixation members can be from about 0.025 mm to 0.5 mm. In other embodiments, the length of the fixation members can be about 0.5 mm to 2.0 mm. In some embodiments, the fixation members and/or retention members can include the use of additional expandable wires, struts, supports, clips, springs, glues, and adhesives. Some embodiments may include a vacuum.

FIG. 5L shows one embodiment of the occlusion device 10 having a separate retention structure 72 coupled to a lattice structure 12. The retention structure 72 can be made from a single wire, or may comprise more than one wire. The retention structure 72 can be secured to the lattice structure 12 and/or any layer of the lattice structure 12 by sewing, suturing, welding, mechanical coupling or any technique known in the art. The retention structure 72 includes non-penetrating retention members 14 attached by chevron-shaped struts 78 arranged circumferentially about the device 10. The chevron-shaped struts provide an array of retention members 14 within a circumferential band or zone of the cylindrical contact region 22 that can extend 2.0-20 mm along the length of the device 10. As shown in FIG. 5L, the retention members 14 can be atraumatic hooks. In other embodiments, the retention members 14 may include fixation members and/or any other suitable retention member shapes and/or configurations disclosed herein.

FIG. 5M shows another embodiment of the occlusion device 10 having a lattice structure 12 including three lattices—an anchoring lattice 86, an occlusive braid 88, and a structural braid 90. The anchoring lattice 86 can be a braid having at least two different filaments with different filament diameters such that portions of the larger filaments can be pulled away from the surface of the anchoring lattice 86 to form retention members 14. For example, in some embodiments, the anchoring lattice 86 may comprise two-thirds structural filaments having diameters between 0.001 in to 0.003 in, and one-third anchoring filaments having diameters between 0.003 in to 0.007 in.

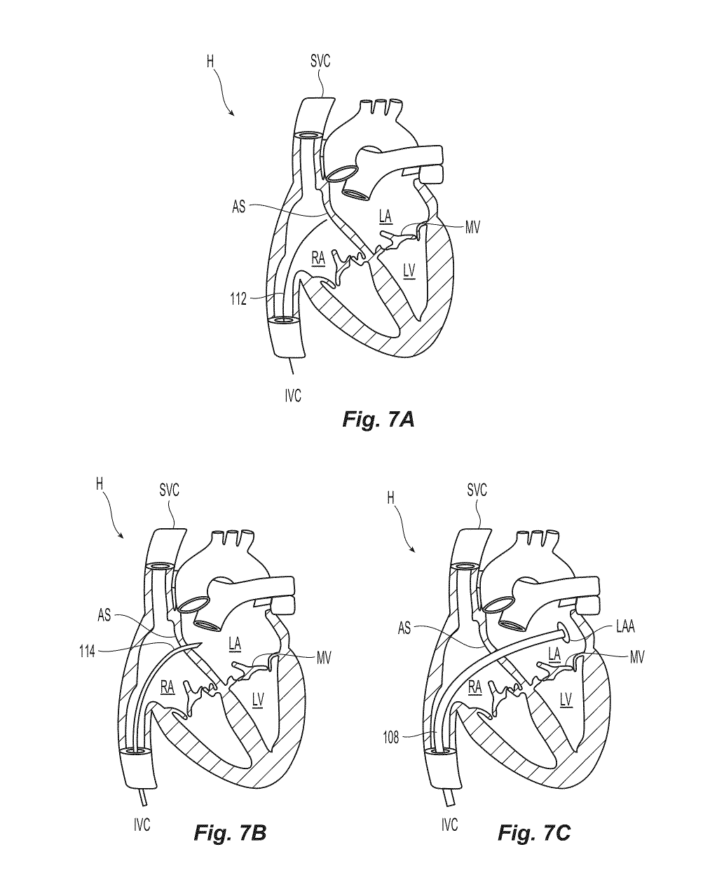

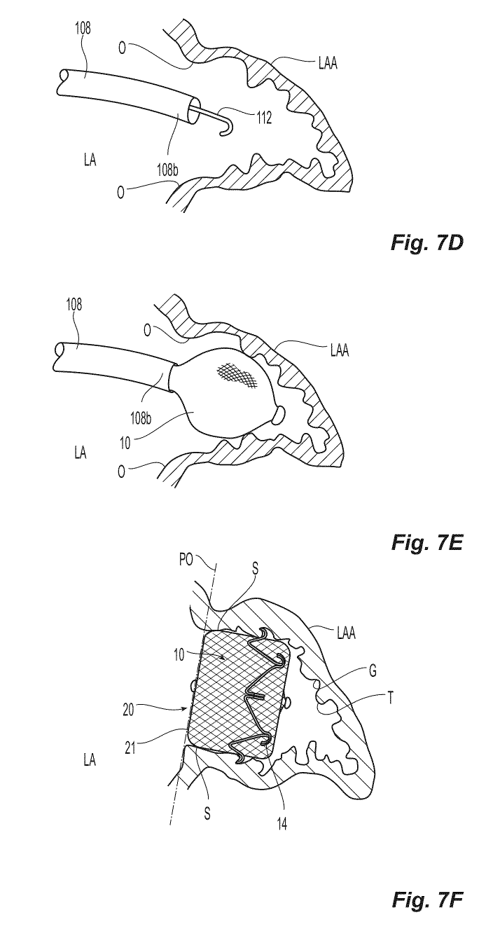

Retention members may be located at any point along the surface of the occlusion device so long as once the device is implanted, at least a portion of the retention members are distal to the smooth entrance region of the LAA (see “S” on FIG. 7F) and positioned to interface with the LAA trabeculae Likewise, the retention members could be in any arrangement (i.e., circumferentially and/or axially, etc.). The retention members and/or retention member associated structures can be constructed using metals, polymers, composites, and/or biologic materials. Polymer materials can include Dacron, polyester, polypropylene, nylon, Teflon, PTFE, ePTFE, TFE, PET, TPE, PLA silicone, polyurethane, polyethylene, ABS, polycarbonate, styrene, polyimide, PEBAX, Hytrel, poly vinyl chloride, HDPE, LDPE, PEEK, rubber, latex, or other suitable polymers. Metal materials can include, but are not limited to, nickel-titanium alloys (e.g. Nitinol), platinum, cobalt-chrome alloys, 35N LT, Elgiloy, stainless steel, tungsten or titanium. In some embodiments, the retention structure 72, retention members 14, occlusive braid 16 and/or structural braid 16 can comprise only metallic materials while the retention structure 72 and/or retention members 14 can be coupled to the occlusive 16 and/or structural braid 18 with a polymeric suture, fastener, or other suitable coupling means known in the art. Accordingly, the occlusion device can be substantially polymer free, that is, polymer free excluding the retention structure and/or retention member coupling means. In yet other embodiments, the occlusion device may not have retention members and is secured to the LAA by the radial and frictional forces of the structural braid 18.

The occlusion device may be constructed to elute or deliver of one or more beneficial drug(s) and/or other bioactive substances into the blood or the surrounding tissue. For example, in some embodiments, the occlusion device may form or contain a reservoir to hold drug(s) and or other bioactive substances, and the occlusion device may include a valve for controlled release of such agents. The reservoir or drug containing portions may be dissolvable or contain dissolving components, including drug and/or structural components. The reservoir can release drugs by elution, diffusion, and/or mechanical actuation or electromechanical devices such as a pressurized gas chamber, a spring release, shape memory release, and/or temperature sensitive release systems.

In some embodiments, the reservoir may be refillable. Refilling drugs and/or actuating a gas or energy source may be by percutaneous hypodermic injection or by an intravascular catheter through a fitting or membrane. In some embodiments, the occlusion device may contain a collapsible reservoir configured to be delivered through an intravascular catheter. After delivery to the LAA, the collapsible reservoir can be expanded and fixed to an interior surface of the LAA.

The drugs and/or bioactive agents include an antiplatelet agent, including but not limited to aspirin, glycoprotein IIb/IIIa receptor inhibitors (including, abciximab, eptifibatide, tirofiban, lamifiban, fradafiban, cromafiban, toxifiban, XV454, lefradafiban, klerval, lotrafiban, orbofiban, and xemilofiban), dipyridamole, apo-dipyridamole, persantine, prostacyclin, ticlopidine, clopidogrel, cromafiban, cilostazol, and nitric oxide. In any of the above embodiments, the device may include an anticoagulant such as heparin, low molecular weight heparin, hirudin, warfarin, bivalirudin, hirudin, argatroban, forskolin, ximelagatran, vapiprost, prostacyclin and prostacyclin analogues, dextran, synthetic antithrombin, Vasoflux, argatroban, efegatran, tick anticoagulant peptide, Ppack, HMG-CoA reductase inhibitors, thromboxane A2 receptor inhibitors, and others.

In some embodiments, the drugs and/or bioactive agents can be release directly into the left atrium. Directly releasing drugs into the heart circulation is advantageous because it requires a lower dose, increases effectiveness, lowers side effects, improves the safety profile, localizes delivery, bypasses the digestive system, substitutes for intravenous or intra-arterial injection, substitutes for oral ingestion, and others. In some embodiments, drug release following implant would be limited to an initial time period of less than five years. In other embodiments, drug release following implant would be limited to an initial time period of less than 1 year. In yet other embodiments, drug release following implant would be limited to an initial time period of less than 3 to 6 months, or in some embodiments, less than 45 days.

In some embodiments, one or more eluting filament(s) may be interwoven into the lattice structure 12 to provide for the delivery of drugs, bioactive agents or materials with a mild inflammatory response as disclosed herein. The interwoven filaments may be woven into the lattice structure after heat treating (as discussed below) to avoid damage to the interwoven filaments by the heat treatment process. In some embodiments, the occlusion device may be coated with various polymers to enhance its performance, fixation and/or biocompatibility. In other embodiments, the device may incorporate cells and/or other biologic material to promote sealing, reduction of leakage and/or healing.

### 2. Delivery Systems and Methods

FIGS. 6A-10B illustrate embodiments of a delivery system 100 and methods for deploying the occlusion device 10. FIG. 6A is a cross-sectional side view of one embodiment of the delivery system 100 showing the occlusion device 10 in a collapsed, low-profile configuration for percutaneous delivery. The delivery system 100 may include a guidewire (not shown), a detachment system 110, and a single or multi-lumen delivery catheter 104 having a proximal hub 106 and a sheath 108. The sheath 108 has a distal zone 108b, a proximal zone 108a, and a lumen therethrough. For example, the lumen of the sheath 108 can have a diameter between 6F and 30F, and in some embodiments, between 8F and 12F.

As shown in FIG. 6B, the detachment system 110 can include a torque cable 102 coupled to screw threads 109 at a distal end of the torque cable 102. The screw threads 109 can match the internal threads of a hole 39 in the locking member 38 of the proximal hub 26 of the device 10 such that unscrewing the screw threads 109 releases the proximal hub 26 from the detachment system 110. In some embodiments, the detachment system may comprise a tether coupled to an electronic system that upon application of an electrical current to the tether severs the tether and releases the device.

Access to the LAA or left atrium LA of the heart can be accomplished through the patient's vasculature in a percutaneous manner. By percutaneous it is meant that a location of the vasculature remote from the heart is accessed through the skin, typically using a surgical cut down procedure or a minimally invasive procedure, such as using needle access through, for example, the Seldinger technique. The ability to percutaneously access the remote vasculature is well-known and described in the patent and medical literature. Once percutaneous access is achieved (for example, through the femoral or iliac veins), the interventional tools and supporting catheter(s) may be advanced to the heart intravascularly and positioned within the LAA in a variety of manners, as described herein.

FIGS. 7A-7F illustrate one example for delivering and deploying an occlusion device 10 and/or one or more interventional devices using an antegrade approach. As shown in FIG. 7A, a guidewire 112 may be advanced intravascularly using any number of techniques, e.g., through the inferior vena cava IVC or superior vena cava SVC (not shown) into the right atrium RA. At this point, the guidewire 112 may be exchanged for a needle 114. As shown in FIG. 7B, the needle 114 punctures the septum AS of the heart to gain access to the left atrium LA. The needle 114 is then removed proximally. Alternatively, the device 10 may be passed through the patent foramen ovale or an existing atrial septal defect to the left atrium LA.

The delivery sheath 108 containing the collapsed occlusion device 10 and detachment system 110 can be advanced together with the guidewire 112 (i.e., using an over the wire or a rapid exchange catheter system) until the distal zone 108b of the catheter is positioned at or distal to the LAA ostium, as shown in FIGS. 7C-7D. The guidewire 112 and catheter 108 can be advanced through the vasculature using known imaging systems and techniques such as fluoroscopy, x-ray, MRI, ultrasound or others. Radiopaque markers (not shown) can be incorporated into the guidewire 112, needle 114, detachment system 110, catheter 104, sheath 108, and/or the occlusion device 10 itself to provide additional visibility under imaging guidance. Such marker materials can be made from tungsten, tantalum, platinum, palladium, gold, iridium, or other suitable materials.

After the distal zone 108b of the sheath 108 is at or proximal to the LAA ostium O, the guidewire 112 is removed proximally through the lumen of the delivery catheter 104. Next, the sheath 108 is retracted proximally and an exposed portion of the occlusion device 10 expands (FIG. 7E) such that a portion of the occlusion device 10 contacts the ostium O and/or LAA wall along at least a portion of a smooth entrance region S of the LAA, as shown in FIG. 7F. In some embodiments, the occlusion device 10 may be actively expanded using conventional techniques known in the art, such as pull-wires attached to a distal end of the device and/or a balloon assembly.

During deployment, the detachment system 110 engages the cap 38 to facilitate deployment of the occlusion device 10. After deployment is completed, the detachment system 110 can disengage from the cap 38 (see FIG. 6B) by unscrewing (i.e., rotating a proximal end of the torque cable 102). In other embodiments, other release mechanisms and/or couplings may be used, including hydraulic, electrothermal, electroresistive, electrolytic, electrochemical, electromechanical and mechanical release mechanisms.

FIG. 7F shows the occlusion device 10 implanted in the LAA with retention members 14 interfacing with the LAA such that the atrial face 21 of the proximal portion 20 of the occlusion device is substantially within or just proximal to the plane of the ostium PO. The fully expanded circumference of the lattice structure 12 may be selected to exceed the circumference of the LAA ostium in order to increase radial force after placement for promoting fixation and sealing. In some embodiments, the maximum expansion of the lattice structure 12 is controlled to expand to the diameter of the LAA.

The LAA often has a “chicken wing” morphology that makes it difficult to properly position, secure and seal existing transcatheter occlusion devices. Just distal to the LAA ostium 0 is a short LAA entrance region S having relatively smooth inner walls. If the proximal end of an occlusion device is positioned too distal to the ostium, the device is likely to turn out of plane of the ostium PO and/or fall deeper into the LAA. Such unwanted repositioning can create a gap between the plane of the ostium PO and the proximal end of the device and/or the proximal end of the device may sit at an angle with respect to the plane of the ostium PO. Such gaps and/or corners/bends/crooks in the device can be potential locations of thrombus formation that defeat the purpose of the occlusion device.

As discussed above with reference to FIGS. 4A-4D, it is often desirable to position the device 10 such that the atrial face 21 is within and/or substantially aligned with the plane of the ostium PO. To facilitate this alignment, the atrial face 21 can be relatively planar or otherwise have a low-profile contour such that it can be positioned substantially flush with the plane of the ostium PO (see FIG. 7F). Since the vast majority of clotting and thrombus formation occurs within the grooves G of the LAA between trabeculae T, positioning the proximal portion 20 of the occlusion device 10 within the smooth entrance region secures and seals the device proximal to the trabeculae T to prevent blood from flowing to the trabeculae T. Also, a device that protrudes too far into the left atrium may disrupt atrial flow, reduce atrial volume, induce high shear forces, promote thrombus and emboli formation, erode tissue, and cause other problems. A device that is positioned too far into the appendage may cause a number of problems including disruption of atrial flow, high shear forces, promotion of thrombus formation, promotion of emboli formation, and others.

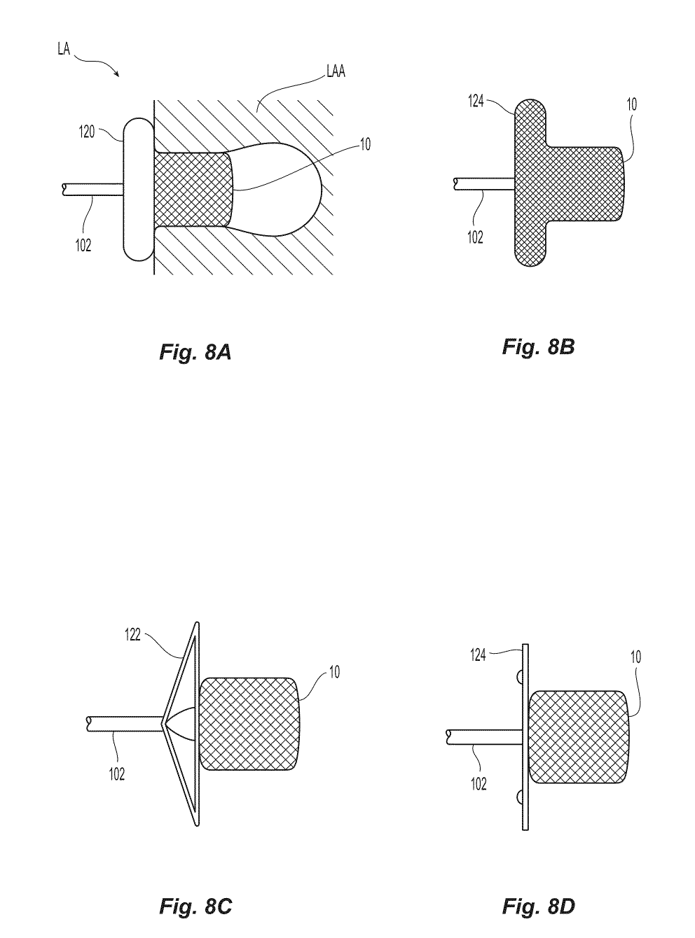

FIGS. 8A-8D show several embodiments in which the delivery system may include one or more positioning members to facilitate positioning a proximal region of the occlusion device 10 in substantial alignment with the plane of the ostium O. For example, as shown in FIG. 8A, the distal region of the delivery system may include a balloon 120 proximal to the occlusion device 10. The balloon 120 can be configured to expand to a diameter greater than the diameter of the LAA ostium such that the balloon 120 abuts the wall of the left atrium LA around the LAA ostium. In some embodiments, the occlusion device is expanded or partially expanded and then the balloon is expanded and positioned against the ostium.

The balloon 120 can be non-compliant or compliant and can have an oblate spheroid, spheroid, spheroid with a flattened side proximate the ostium, or other suitable shapes. In one embodiment, the occlusion device 10 and balloon 120 are inserted intravascularly to the left atrium and initially positioned inside the LAA using imaging modalities including TEE, fluoroscopy, CT, and others. The balloon may be filled with a contrast medium to aid in visualization and/or radiopaque markers may be placed on the balloon, catheter or occlusion device to aid in visualization before, during and after placement. The balloon is deflated prior to removal from the left atrium. In some embodiments, other positioning structures may be used in addition to or in place of the balloon, including an expandable braided mesh (FIG. 8B), an expandable Malecot structure (FIG. 8C), a mechanical positioner (FIG. 8D), or other suitable positioning structures.

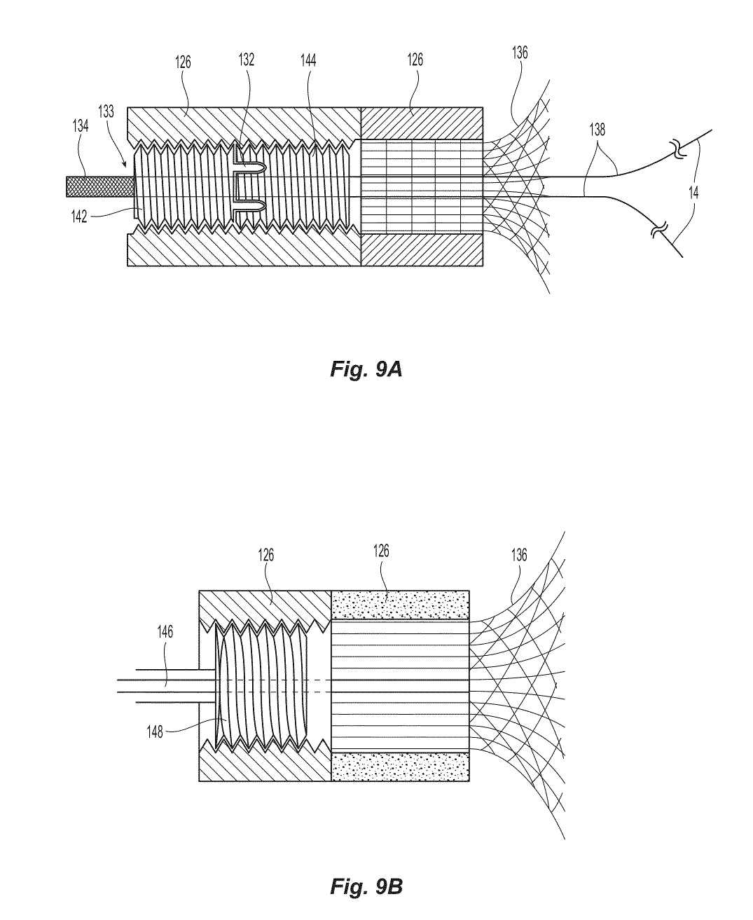

FIG. 9A shows a cross-sectional side view of one embodiment of an occlusion device delivery system having an actuator 133 for deploying retention members 14 (e.g., atraumatic or traumatic retention members as shown in FIGS. 4A and 4B). The actuator 133 can include a rod or torque cable 134 connected to a threaded head 142 that interfaces with a threaded traveler 144. The threaded head 142 can have pins 132 at a distal end configured to mate with corresponding holes in the threaded traveler 144. The threaded traveler 144 can be coupled to wires 138 which extend distally through the lattice structure 136 and are coupled to or integrated with retention members 14. As the rod 134 rotates, the threaded head 142 and the threaded traveler 144 move proximally. The proximal movement of the threaded traveler 144 pulls the wires 138 to deploy the retention members outwardly relative to the lattice 12 (FIGS. 4A and 4B). Once the threaded head 142 clears the proximal hub 126, the occlusion device is released.

FIG. 9B shows another embodiment of an actuator where a rod or torque cable 146 extends distally through a proximal hub 126 of a lattice structure 136 and is coupled to retention members (not shown). Proximal and/or distal movement of the rod 146 can actuate the retention members so as to interface with LAA tissue.

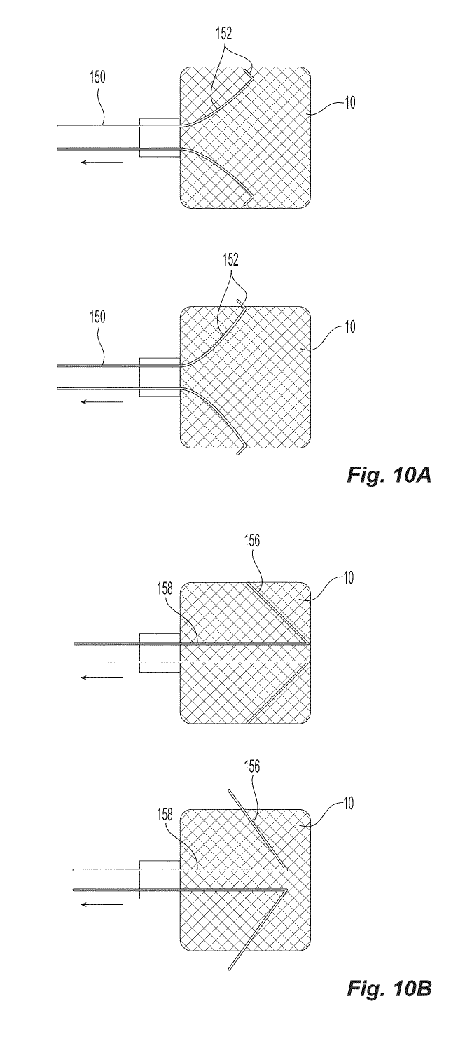

FIGS. 10A-10B illustrate various retention member (traumatic or atraumatic) actuation mechanisms in accordance with embodiments of the present technology. FIG. 10A shows proximal movement of retention member wires 150 can cause retention members 152 to catch on a ramp, post and/or guide structure, causing the retention members 152 to bend in a proximal direction and/or expand radially so as to engage LAA tissue. FIG. 10B shows proximal movement of retention member wires 158 can cause retention members 156 to catch on a ramp, post and/or guide structure, causing the retention members 156 to bend in a proximal direction and/or expand radially. In some embodiments, the retention members 152 or 156 can already be partially protruding when proximal motion begins, such that the retention members catch on the occlusion device 10.

### 3. Lattice Structure and Formation

In any of the embodiments described herein, the lattice structure and/or layers comprising the lattice structure can be a latticework, mesh, and/or braid of wires, filaments, threads, sutures, fibers or the like, that have been configured to form a fabric or structure having openings (e.g., a porous fabric or structure). The mesh can be constructed using metals, polymers, composites, and/or biologic materials. Polymer materials can include Dacron, polyester, polypropylene, nylon, Teflon, PTFE, ePTFE, TFE, PET, TPE, PLA silicone, polyurethane, polyethylene, ABS, polycarbonate, styrene, polyimide, PEBAX, Hytrel, poly vinyl chloride, HDPE, LDPE, PEEK, rubber, latex, or other suitable polymers. Other materials known in the art of elastic implants can also be used. Metal materials can include, but are not limited to, nickel-titanium alloys (e.g. Nitinol), platinum, cobalt-chrome alloys, 35N LT, Elgiloy, stainless steel, tungsten or titanium. In certain embodiments, metal filaments may be highly polished or surface treated to further improve their hemocompatibility. In some embodiments, it is desirable that the mesh be constructed solely from metallic materials without the inclusion of any polymer materials, i.e., polymer free. In these embodiments and others, it is desirable that the entirety of the occlusion device be made of metallic materials free of any polymer materials. It is believed that the exclusion of polymer materials in some embodiments may decrease the likelihood of thrombus formation on device surfaces, and it is further believed that the exclusion of polymers and the sole use of metallic components can provide an occlusion device with a thinner profile that can be delivered with a smaller catheter as compared to devices having polymeric components.

FIG. 11A shows the lattice structure and/or lattices comprising the lattice structure being formed over a mandrel 160 as is known in the art of tubular braid manufacturing. The braid angle alpha a can be controlled by various means known in the art of filament braiding. The tubular braided mesh can then be further shaped using a heat setting process. Referring to FIG. 11A, as is known in the art of heat setting a braiding filament, such as Nitinol wires, a fixture, mandrel or mold can be used to hold the braided tubular structure in its desired configuration while subjected to an appropriate heat treatment such that the resilient filaments of the braided tubular member assume or are otherwise shape-set to the outer contour of the mandrel or mold. The filamentary elements of a mesh device or component can be held by a fixture configured to hold the device or component in a desired shape and, in the case of Nitinol wires, heated to about 475-525° C. for about 5-30 minutes to shape-set the structure. Such braids of shape memory and/or elastic filaments are herein referred to as “self-expanding.” Other heating processes are possible and will depend on the properties of the material selected for braiding.

For braided portions, components, or elements, the braiding process can be carried out by automated machine fabrication or can also be performed by hand. For some embodiments, the braiding process can be carried out by the braiding apparatus and process described in U.S. Pat. No. 8,261,648, filed Oct. 17, 2011 and entitled “Braiding Mechanism and Methods of Use” by Marchand et al., which is herein incorporated by reference in its entirety. In some embodiments, a braiding mechanism may be utilized that comprises a disc defining a plane and a circumferential edge, a mandrel extending from a center of the disc and generally perpendicular to the plane of the disc, and a plurality of actuators positioned circumferentially around the edge of the disc. A plurality of filaments are loaded on the mandrel such that each filament extends radially toward the circumferential edge of the disc and each filament contacts the disc at a point of engagement on the circumferential edge, which is spaced apart a discrete distance from adjacent points of engagement. The point at which each filament engages the circumferential edge of the disc is separated by a distance “d” from the points at which each immediately adjacent filament engages the circumferential edge of the disc. The disc and a plurality of catch mechanisms are configured to move relative to one another to rotate a first subset of filaments relative to a second subset of filaments to interweave the filaments. The first subset of the plurality of filaments is engaged by the actuators, and the plurality of actuators is operated to move the engaged filaments in a generally radial direction to a position beyond the circumferential edge of the disc. The disc is then rotated a first direction by a circumferential distance, thereby rotating a second subset of filaments a discrete distance and crossing the filaments of the first subset over the filaments of the second subset. The actuators are operated again to move the first subset of filaments to a radial position on the circumferential edge of the disc, wherein each filament in the first subset is released to engage the circumferential edge of the disc at a circumferential distance from its previous point of engagement.

In some embodiments, the lattice structure and/or layers of the lattice structure may be formed using conventional machining, laser cutting, electrical discharge machining (ECM) or photochemical machining (PCM). In some embodiments, the lattice structure and/or layers of the lattice structure may be formed from metallic tubes and/or sheet material. Some PCM processes for making similar structures are described in U.S. Pat. No. 5,907,893, filed Jan. 31, 1997 entitled “Methods for the Manufacture of Radially Expansible Stents” by Zadno-Azizi et al., and in U.S. Pat. No. 7,455,753, filed Oct. 10, 2006 entitled “Thin Film Stent” by Roth, which are both herein incorporated in their entirety by reference.

The terms “formed,” “preformed,” and “fabricated” may include the use of molds or tools that are designed to impart a shape, geometry, bend, curve, slit, serration, scallop, void, hole in the elastic, superelastic, or shape memory material or materials used in the components of the occlusion device, including the mesh. These molds or tools may impart such features at prescribed temperatures or heat treatments.

The filaments of the braids can be arranged in a generally axially elongated configuration when the occlusion device 10 is within the delivery catheter. In the expanded or deployed configuration, certain embodiments of the filaments have a “low” filament braid angle “a” from about 5 to 45 degrees with respect to the longitudinal axis of the device such that the filaments are angled toward the longitudinal dimension of the occlusion device 10. In some embodiments, the filaments can have a “high” braid angle a between about 45 to 85 degrees with respect to the longitudinal axis of the occlusion device. The braids for the mesh components can have a generally constant braid angle a over the length of a component or can be varied to provide different zones of pore size and radial stiffness. The expanded braided mesh can conform to or otherwise contact the vessels without folds along the longitudinal axis. The cross-sectional dimension of the lattice structure in the expanded state can be from 3 mm to 60 mm, or from 10 mm to 40 mm in some embodiments. The diameters of the lattice structure within the delivery catheter can be about 1 mm to 15 mm, or 5 mm to 10 mm in more specific applications.

As shown in FIG. 11B, in some embodiments, braid filaments of varying diameters may be combined in the same layer of the lattice or portions of the lattice to impart different characteristics including, e.g., stiffness, elasticity, structure, radial force, pore size, embolic filtering ability, and/or other features. For example, in the embodiment shown in FIG. 11 B, the braided mesh has a first mesh filament diameter 164 and a second mesh filament diameter 164 smaller than the first mesh filament diameter 164. In some embodiments, the diameter of the structural 18 and/or occlusive 16 braid filaments can be less than about 0.5 mm. In other embodiments, the filament diameter may range from about 0.01 mm to about 0.40 mm. In some embodiments, the thickness of the structural braid 18 filaments would be less that about 0.5 mm. In some embodiments, the structural braid 18 may be fabricated from wires with diameters ranging from about 0.015 mm to about 0.25 mm. In some embodiments, the thickness of the occlusive braid 16 filaments would be less that about 0.25 mm. In some embodiments, the occlusive braid 16 may be fabricated from wires with diameters ranging from about 0.01 mm to about 0.20 mm.

As used herein, “pore size” refers to the diameter of the largest circle 162 that fits within an individual cell of a braid (see FIG. 11 B). The average and/or maximum pore size of the structural braid 18 can be greater than 0.20 mm, and generally more than 0.25 mm. The structural braid 18 or portions of the structural braid 18 are configured to provide stability and exert radial forces that secure and shape other layers and/or braids of the lattice structure 12 to surrounding tissue structures. The radial force exerted by the structural braid 18 is generally sufficient to inhibit movement, dislodgement and potential embolization of the occlusion device 10. For the occlusive braid 16, average and/or maximum pore sizes in the range of about 0.025 mm to 2.0 mm may be utilized. In some embodiments, the occlusive braid 16 average and/or maximum pore sizes may be in the range of 0.025 mm to 0.300 mm, outside the range of existing devices. Likewise, the radial stiffness of the structural braid 18 can be 10-100 times greater than the radial stiffness of the occlusive braid 16. In some embodiments, the radial stiffness of the structural braid 18 is 10-50 times greater than the radial stiffness of the occlusive braid 16.

Different layers of the lattice structure 12 may have different filament counts. In some embodiments, the braided filament count for the occlusive braid 16 is greater than 290 filaments per inch. In one embodiment, the braided filament count for the occlusive braid 16 is between about 360 to about 780 filaments, or in further embodiments between about 144 to about 290 filaments. In one embodiment, the braided filament count for the structural braid 18 is between about 72 and about 144 filaments, or in other embodiments between about 72 and about 162 filaments. In some embodiments, the device 10 may include polymer filaments or fabric within the lattice layers 16, 18 or between layers of braids.

For some embodiments, three factors are often desirable for a woven or braided wire occlusion device that can achieve a desired clinical outcome in the endovascular treatment of LAA. For effective use in some applications, it may be desirable for the occlusion device to have sufficient radial stiffness for stability, limited pore size for rapid promotion of hemostasis leading to occlusion, and a collapsed profile which is small enough to allow insertion through an inner lumen of a vascular catheter. A device with a radial stiffness below a certain threshold may be unstable and may be at higher risk of movement or embolization in some cases. Larger pores between filament intersections in a braided or woven structure may not generate thrombi and cause occlusion in an acute setting and thus may not give a treating physician or health professional such clinical feedback that the flow disruption will lead to a complete and lasting occlusion of the LAA being treated. Delivery of a device for treatment of a patient's vasculature through a standard vascular catheter may be highly desirable to allow access through the vasculature in the manner that a treating physician is accustomed. The “average maximum pore size” in a portion of a device that spans the LAA ostium is desirable for some useful embodiments of a braided wire device for treatment and may be expressed as a function of the total number of all filaments, filament diameter and the device diameter. As used in the equation below and accompanying discussion, “average maximum pore size” refers to an average pore size of the “M” largest pore sizes in the portion of the device that spans the LAA ostium, where M is a positive integer that varies based on the device. For example, in some devices, it may be appropriate to select an M of 10. In this case, the ten largest pore sizes in the portion of the device that spans the LAA ostium would be averaged to determine the average maximum pore size in that portion of the device. The difference between filament sizes, where two or more filament diameters or transverse dimensions are used, may be ignored in some cases for devices where the filament size(s) are very small compared to the device dimensions. For a two-filament device, the smallest filament diameter may be used for the calculation. Thus, the average maximum pore size for such embodiments may be expressed as follows:

Pₘₐₓ=(1.7/NT)*(pD−(NTdw/2));

where Pₘₐₓ is the average maximum pore size;

D is the device diameter (transverse dimension);

NT is the total number of all filaments; and

dw is the diameter of the filaments (smallest) in inches.

Using this expression, the average maximum pore size, Pₘₐₓ, of the of the device may be less than about 0.016 inches or about 400 microns for some embodiments. In some embodiments the average maximum pore size of the device may be less than about 0.012 inches or about 0.300 mm. In some embodiments, the average maximum pore size of the device can be between 0.1 mm to 0.3 mm. In other embodiments, the average maximum pore size of the device can be between 0.075 mm to 0.250 mm.

The collapsed profile of a two-filament (profile having two different filament diameters) braided filament device may be expressed as the function:

P_{c}=1.48((Nₗdₗ²+Nₛdₛ²))^{1/2}

where P_{c} is the collapsed profile of the device;

Nₗ is the number of large filaments;

Nₛ is the number of small filaments;

dₗ is the diameter of the large filaments in inches; and

dₛ is the diameter of the small filaments in inches.

Using this expression, the collapsed profile P_{c} may be less than about 4.0 mm for some embodiments of particular clinical value. In some embodiments of particular clinical value, the device may be constructed so as to have both factors (Pₘₐₓ and P_{c}) above within the ranges discussed above; Pₘₐₓ less than about 300 microns and P_{c} less than about 4.0 mm, simultaneously. In some such embodiments, the device may be made to include about 200 filaments to about 800 filaments. In some cases, the filaments may have an outer transverse dimension or diameter of about 0.0008 inches to about 0.012 inches.

In some embodiments, a combination of small and large filament sizes may be utilized to make a device with a desired radial compliance and yet have a collapsed profile which is configured to fit through an inner lumen of commonly used vascular catheters. A device fabricated with even a small number of relatively large filaments can provide reduced radial compliance (or increased stiffness) compared to a device made with all small filaments. Even a relatively small number of larger filaments may provide a substantial increase in bending stiffness due to change in the moment of Inertia (I) that results from an increase in diameter without increasing the total cross sectional area of the filaments. The moment of inertia (I) of a round wire or filament may be defined by the equation:

I=πd⁴

where d is the diameter of the wire or filament.

Since the moment of inertia is a function of filament diameter to the fourth power, a small change in the diameter greatly increases the moment of inertia. Thus, a small change in filament size can have substantial impact on the deflection at a given load and thus the compliance of the device.

Thus, the stiffness can be increased by a significant amount without a large increase in the cross sectional area of a collapsed profile of the device. This may be particularly important as device embodiments are made larger to treat larger LAA. As such, some embodiments of devices for treatment of a patient's vasculature may be formed using a combination of filaments with a number of different diameters such as 2, 3, 4, 5 or more different diameters or transverse dimensions. In device embodiments where filaments with two different diameters are used, some larger filament embodiments may have a transverse dimension of about 0.004 inches to about 0.012 inches and some small filament embodiments may have a transverse dimension or diameter of about 0.0008 inches and about 0.003 inches. The ratio of the number of large filaments to the number of small filaments may be between about 4 to 16 and may also be between about 6 to 10. In some embodiments, the difference in diameter or transverse dimension between the larger and smaller filaments may be less than about 0.008 inches. In some embodiments, less than about 0.005 inches, and in other embodiments, less than about 0.003 inches.

For some embodiments, it may be desirable to use filaments having two or more different diameters or transverse dimensions to form a permeable shell in order to produce a desired configuration as discussed in more detail below. The radial stiffness of a two-filament (two different diameters) woven device may be expressed as a function of the number of filaments and their diameters, as follows:

S_{radial}=(1.2×10⁶ lbf /D⁴)*(Nₗdₗ⁴+Nₛdₛ⁴);

where S_{radial} is the radial stiffness in pounds force (lbf);

D is the device diameter (transverse dimension);

Nₗ is the number of large filaments;

Nₛ is the number of small filaments;

dₗ is the diameter of the large filaments in inches; and

dₛ is the diameter of the small filaments in inches.

Using this expression, the radial stiffness, S_{radial} may be between about 0.014 and 0.284 lbf force for some embodiments of particular clinical value.

### 4. Occlusion Device Shapes and Layering

The occlusion device can have various geometries depending on the application. For example, the occlusion device can include one or more layers of the same lattice material or different lattice materials that have a generally cylindrical, spherical, ellipsoidal, oval, barrel-like, conical, frustum or other geometric shape. The lattice layers or portions of the lattice layers can have an undulated or wave-like contour, a saw-toothed contour, a bellows-like contour, a sinusoidal contour, and/or other suitable surface contours. Other suitable occlusion devices and/or lattice structures are disclosed in PCT Application No. PCT/US12/51502 filed Aug. 17, 2012, entitled “EXPANDABLE OCCLUSION DEVICES AND METHODS,” the full disclosure of which is incorporated by reference.

The lattice structure of the occlusion device can have one or more braided or mesh layer. Two layers can be formed from one tubular braid that has been everted or folded back on itself to form a two-layer construct as describe above with regard to FIGS. 4A-4D. An everted lattice forming two layers can be either the innermost layers, intermediate layers or outermost layers of the lattice structure. In some embodiments, the layers can be configured in a substantially coaxial fashion. In other embodiments, the layers or some of the layers can be held at one or more ends by a common connecting member or hub. In some embodiments, one or more of the layers can have an open end that is not held by a connecting member or hub. An unfixed end of the layer can allow the individual layers to have different lengths without bunching of the layers upon collapse for delivery or retraction by a catheter because the free ends of the layers can move relative to each other to accommodate the compression of the occlusion device into a contracted state.

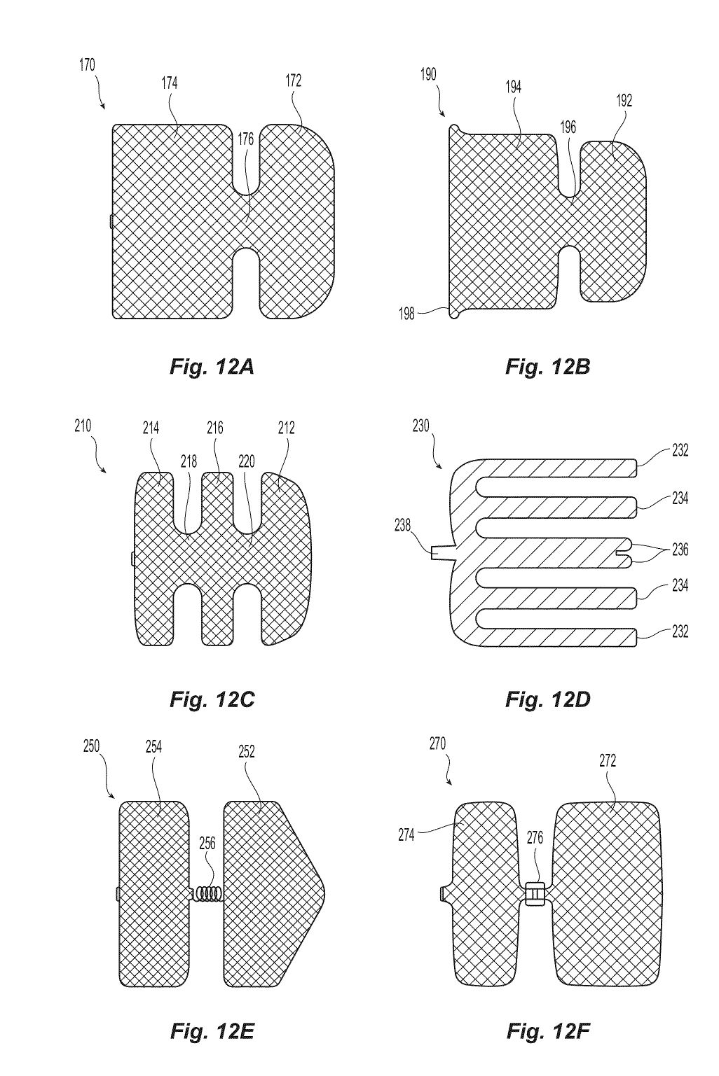

Several configurations of occlusion devices and/or lattice structure shapes are described in the following embodiments. As can be appreciated, the described features or combination of features for a particular embodiment can be applied to another embodiment. Furthermore, for clarity, features that are common to earlier-described embodiments are not again described in detail with reference to FIGS. 12A-14 as reference can be made to those features in earlier descriptions. For example, although only the outermost layer is shown in the lattice structures illustrate in FIGS. 12A-14, any of the lattice structure sections described below can comprise one or more braided layers along its entire length or a portion of its length.

FIG. 12A illustrates an embodiment of a lattice structure 170 having a proximal section 174 and a distal section 172 connected to the proximal section 174 by a connecting section 176. The proximal section 174 fixates and seals the device 170 to the ostium and/or LAA while the distal section 172 extends into the LAA cavity and further fixates the device. The connecting section 176 facilitates flexing of the lattice structure 170 along its central longitudinal axis so as to adjust to one or more lobes of the LAA. In some embodiments the proximal and/or distal sections 174 and 172 can have an oval shape or other shapes to conform to the geometry of the LAA ostium and appendage body.

In some embodiments, the radial stiffness of the distal section may be substantially less than the radial stiffness of the proximal section. Accordingly, the distal section may be much more compliant than the proximal section to conform to anatomical variations often found in the LAA. The malleability of the distal section improves surface area contact with the LAA walls and/or trabeculae and resists movement. In some embodiments, the radial stiffness of the proximal section may be between about 1.5 times to 5 times the radial stiffness of the distal section.

Referring to FIG. 12B, the lattice structure can have a flange 198 at a proximal edge of the proximal section 194. When deployed, the flange 198 is positioned in contact with the left atrium wall at or slightly proximal to the ostium of the LAA. The flange 198 is expected to align the proximal face of the device 10 with the plane of the LAA ostium. This may assist in preventing the device 10 from turning out of the plane of the LAA ostium.

In other embodiments, the lattice structure can have more than two lattice sections. For example, FIG. 12C shows one embodiment of an occlusion device having a proximal section 214, a middle section 216, and a distal section 212. The proximal section 214 connects to the middle section 216 through a first connector 218, and the middle section connects to the distal section through a second connector 220. FIG. 12D shows another embodiment of a lattice structure 230 having a plurality of annular lattice sections including, for example, an outer ring 232, an intermediate ring 234, and an inner ring 236.

In some embodiments, the sections of the lattice structure may be coupled by a connector. For example, as shown in FIG. 12E, a lattice structure 250 can have a proximal section 254 and a distal section 252 coupled by a spring 256. In other embodiments, the connector can be a mechanical coupling 276, as shown in FIG. 12F.

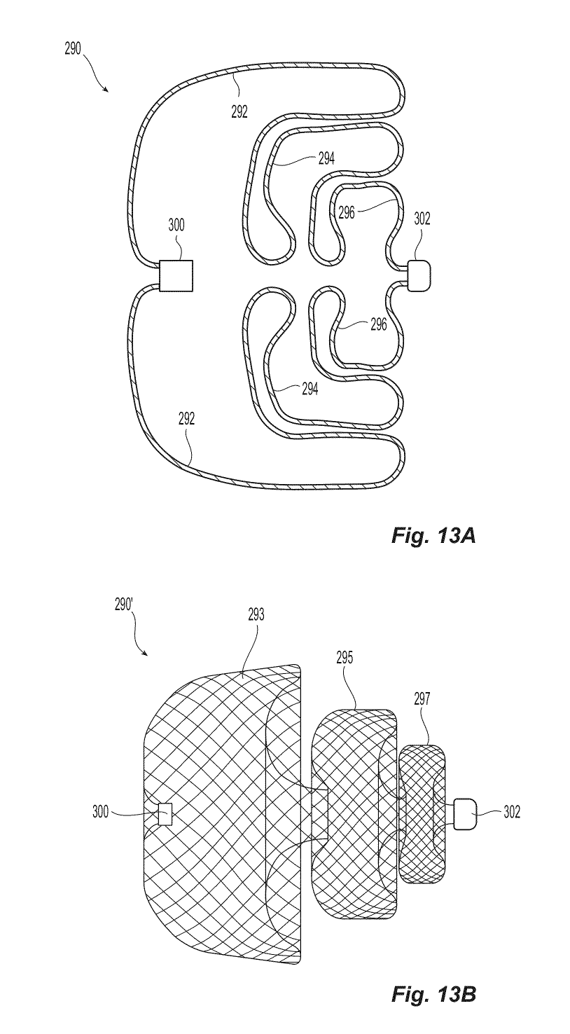

Referring to FIGS. 13A-13B, in some embodiments the lattice structure may have nested sections. As shown in the cross-sectional side view of FIG. 13A, a lattice structure 290 can comprise a single lattice having two drooping sections, 292 and 294, and a third section 296. The two drooping sections 292 and 294 can be angled to have a dog-legged shape. The single lattice is secured at a proximal end to a proximal hub 300 and secured at a distal end to a distal hub 302. The outer section 292 at least partially encompasses an intermediate section 294 and the intermediate section 294 at least partially encompasses an inner section 296. The outer section 292 can define a proximal portion of the lattice structure 290, while all three sections can define a distal portion of the lattice structure 290. FIG. 13B is a schematic side view of the nested lattice structure 290 when slight tension is applied in opposite directions to the hubs 300 and 302 (i.e., stretched out).

It will be appreciated that specific elements, substructures, advantages, uses, and/or other features of the embodiments described with reference to FIGS. 12A-13B can be suitably interchanged, substituted or otherwise configured with one another and/or with the embodiments described with reference to FIGS. 3-11B in accordance with additional embodiments of the present technology. For example, although the lattice structure of FIG. 12C is shown having mesh connectors 218 and 220, the spring coupling 256 from FIG. 12E may be substituted for mesh connectors 218 and 220. Furthermore, suitable elements of the embodiments described with reference to FIGS. 12A-13B can be used as standalone and/or self-contained devices.

Many of the foregoing devices, as well other devices, include more than one layer of braided wire structure, or a “multilayered braid.” It is often necessary to compensate for differences in the surface areas of such multilayered braided structures for movement between a low-profile configuration for delivery via a catheter to an expanded deployed configuration for implantation by adjusting the pick angle of the different braided structure layers. This includes three dimensional braided wire structures that are intended to be deformed into generally cylindrical, minimum diameter structures in order to fit into minimum diameter delivery catheters to facilitate placement through blood vessels and/or other anatomical structures or orifices. These devices may expand when exiting the delivery catheter. Some embodiments may include devices for: left atrial appendage occlusion, atrial septal defect occlusion, ventricular septal defect occlusion, patent foramen ovale occlusion, patent ductus arteriosus occlusion, aneurysm occlusion, neurovascular aneurysm occlusion, thoracic aortic aneurysm grafts, thoracic aortic aneurysm dissection grafts, peripheral vascular grafts, coronary stents, coronary vascular grafts, cardiovascular shunts, ophthalmological shunts, and shunts for other anatomical structures in the body, abdominal aortic aneurysm grafts, abdominal aortic dissection grafts, vascular occlusion, cardiovascular defects, congenital defects and other anatomical defects. Other embodiments may include: cerebral embolic protection, thrombectomy, introduction sheaths, catheters, and guiding catheters.

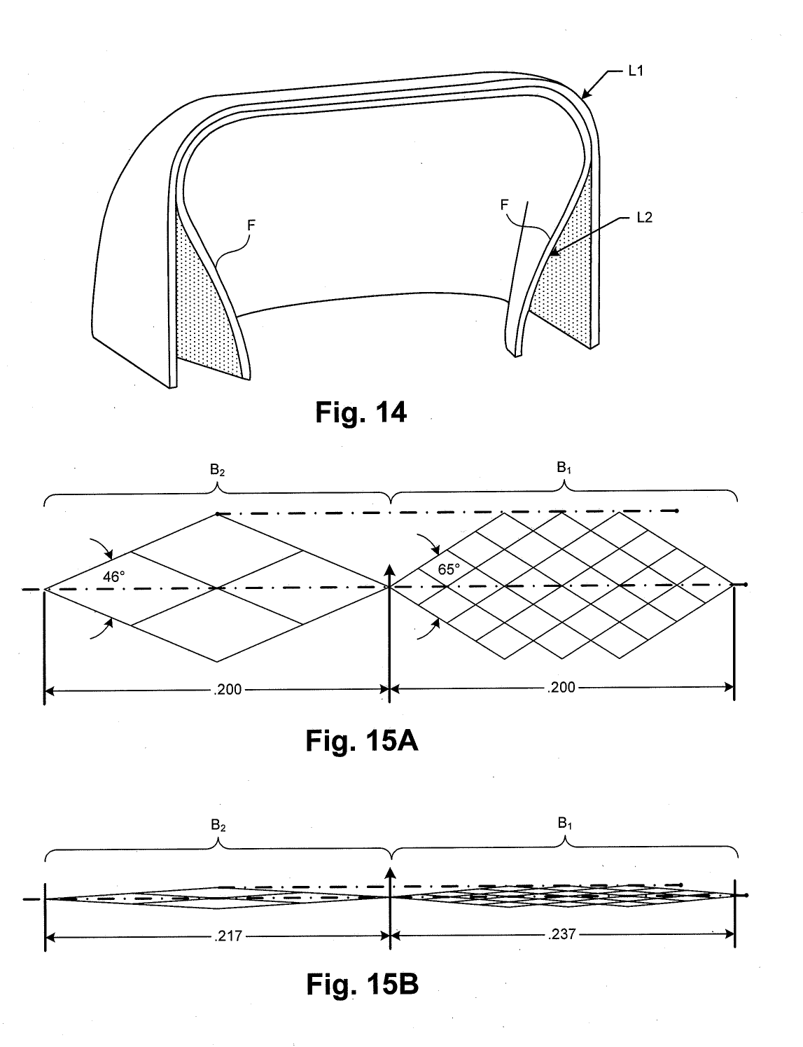

FIG. 14 is a cross-sectional view of a portion of a multilayered braided device, and FIGS. 15A and 15B are diagrams of the relationship between the pick angle of a braid and its length in an expanded state (FIG. 15A) and a contracted state (FIG. 15B). FIG. 14 shows a portion of a multilayered braided device 1400 having an outer braid layer (L1) and an inner braid layer (L2). As described above, the outer braid layer (L1) can be an occlusion braid having small wires and small pore sizes, and the inner braid layer (L2) can be a structural braid having larger wires and larger pore sizes. The inner braid layer (L2) can have a inwardly contoured portion (F) that allows the outer braid layer (L1) to expand to the desired diameter in the deployed state. However, to collapse the device 1400 from the expanded state shown in FIG. 14 to a fully contracted state, the outer braid layer (L1) must extend longitudinally to a greater extent that the inner braid layer (L2) to compensate for the extra length of the material of the inner braid layer (L2) due to the inwardly contoured portion (F).

One way to accomplish this is to make the outer braid layer (L1) from a braid having a first pick angle and the inner braid layer (L2) from a braid having a second pick angle less than the first pick angle. For example, referring to FIGS. 15A and 15B, these figures show the different properties of a first braid portion B₁ having a pick angle of 65° and a second braid portion B₂ having a pick angle of 46°. The first and second braid portions B₁ and B₂ can be different braids (e.g., different braided structures or different layers of braids). FIG. 15A shows the first and second braid portions B₁ and B₂ in an expanded state in which both have a length of 0.200, and FIG. 15B shows the first and second braid portions B₁ and B₂ in a contracted state in which the first braid portion B₁ has a length of 0.237 and the second braid portion B₂ has a length of 0.217. Thus, referring to FIG. 14, the outer braid layer (L1) can have a first braid with a first pick angle and the inner braid layer (L2) can have a second braid with a second braid angle less that the first braid angle such that the outer braid layer (L1) extends further from the expanded state to the collapsed state so that the inner braid layer (L2) can extend to its full length in the collapsed state (e.g., extend to a length without a inwardly contoured portion).

As noted above, braided tubes can be formed into closed three dimensional structures by reducing the diameter of both ends of the tube until the all of the filaments of the braid are touching. Such three dimensional structures have an axis determined by the center of the reduced diameter ends. The maximum diameter of the three dimensional structure is determined by the pick angle (the helix angle of the filament from a line on the braid surface parallel to the axis) and outside diameter of the parent tubular braid. The minimum diameter of the reduced diameter ends is determined by the number and diameter of the braid filaments (assuming folding of the braid wall).

The bounded area between 4 braid filaments is called a pore. For example, the pores in FIGS. 15A and 15B are the diamond-shaped structures. The number of pores is a fixed value determined by the number of filaments, the pick angle, and the braid length. The area of a pore varies with the pick angle. Therefore, the surface area of a braid is not constant as the diameter is reduced and the length increased.

If the filaments are made of a sufficiently resilient material, the three dimensional structure can be deformed into a minimum diameter solid cylinder on the same axis. The length of this cylinder is somewhat less than the length of an individual filament.

The minimum area surface that can encompass any volume is spherical. Dimpling a sphere reduces its volume. Therefore, scaling the dimpled sphere to match the original volume of the sphere results in a shape that has a larger surface area than the original sphere. Similarly, dimpling the ends of a right cylinder reduces its volume while increasing its surface area.

A right cylinder can be constructed from a braid by gathering the ends to the minimum diameter. The braid filaments are flexible, but do not elongate under typical use conditions. If an end of the cylinder is dimpled, the filament length must be increased to follow the new surface. In other words: the deeper the dimple, the larger the surface area and the longer the required filament length. Therefore, if a braided dimpled cylinder is collapsed to its minimum diameter, the resultant solid cylinder will be longer than a collapsed braided right cylinder. This assumes that the pick angles of the parent braids were the same for both cylinders.

It is often advantageous to develop structures made up of more than one braided tube. Inner braided layers may be made of fewer, large diameter wires that provide structural, “skeletal,” support to the structure. Outer braided layers may be made of more numerous small diameter wires that result in many, very small, pores. These layers could provide a “skin” and barrier characteristics but would have little structural integrity on their own.

For embodiments composed of multiple braided layers forming a closed shape that need to be deformed—as in for deployment into a cavity through a small orifice—the collapsed length of the outer layers must be longer than that of the inner layers or the structure will not completely collapse for the reasons explained above. Additionally, the structural layers may include features that increase their surface area (i.e. dimples or convolutions). If the “skin” layers do not conform to the structural layers (possibly formed as a minimum surface encompassing the structural layers) the surface area of the “skin” layers can be substantially less than that of the structural layers. For the complete structure to deform properly, the outer layers must elongate proportionately farther than the inner layers.

This accommodation is provided by the pick angle of the parent braids of the outer layers being larger than that of the inner layers as shown FIGS. 14, 15A and 15B.

From the foregoing, it will be appreciated that specific embodiments of the invention have been described herein for purposes of illustration, but that various modifications may be made without deviating from the scope of the invention.

I/we claim:

1. A device comprising at least an outer braided structure having a first braid with a first pick angle and an inner braided structure having a second braid with a second pick angle, wherein the first pick angle of the outer braided structure is greater than the second pick angle of the inner braided structure.

2. A device comprising at least an outer layer having a first braid with a first length and an inner layer having a second braid with a second length, and wherein the first length of the first braid is greater than the second length of the second braid.

3. A device comprising an inner structural braid layer and an outer occlusive braid layer, wherein a pick angle of the structural braid layer is less than a pick angle of the occlusive braid layer.

4. A device comprising an expandable lattice structure including:
an occlusive braid configured to contact and seal with tissue;
a structural braid enveloped by the occlusive braid;
wherein the structural braid is configured to drive the occlusive braid outward to press the occlusive braid against the tissue, and wherein the occlusive braid has a first pick angle greater and the structural braid has a second pick angle less than the first pick angle.

5. A device comprising at least two layers of braided structure, wherein a lengthening of the device causes a reduction in a diameter of the device from an expanded state to a contracted state, and wherein a pick angle of each outer layer is larger than a pick angle of an adjacent inner layer.

6. A device comprising two or more braid layers, wherein a lengthening of the device causes:
a reduction in a diameter of the device from an expanded state to a contracted state;
wherein the reduction in diameter is sufficient to position the device within an elongate delivery catheter to an anatomic site; and
wherein a pick angle of one braid layer is larger than a pick angle of a successive inner braid layer.

7. A device comprising two or more braid layers wherein deployment of the device from a contracted state within a catheter results in a diameter expansion of the device of a least 50% and wherein a pick angle of an outer braid layer is larger than a pick angle of a braid layer within the outer layer.

8. A device comprising an expandable lattice structure that comprises:
an occlusive braid configured to contact and seal with tissue within a body structure;
a structural braid enveloped by the occlusive braid and couple to the occlusive braid at a proximal hub located at the proximal region of the lattice structure; and
wherein the structural braid is configured to drive the occlusive braid radially outward to press the occlusive braid against at least a portion of the tissue of the body structure and the pick angle of the occlusive braid is greater than the pick angle of the structural braid.

9. A device with an expandable lattice structure that comprises:
an occlusive braid having a first pick angle configured to contact and seal with tissue within a body structure;
a structural braid having second pick angle enveloped by the occlusive braid and couple to the occlusive braid at a proximal hub located at the proximal region of the lattice structure; and
wherein the structural braid is configured to drive the occlusive braid radially outward to press the occlusive braid against at least a portion of the tissue of the body structure, and the first pick angle of the occlusive braid is greater than the second pick angle of the structural braid; and
wherein the occlusive braid has a first radial stiffness and the structural braid has a second radial stiffness that is 10 to 100 times greater than the first radial stiffness.

10. A device comprising an expandable lattice structure that comprises:
an occlusive braid configured to contact and seal with tissue, wherein the occlusive braid has a first pick angle;
a structural braid enveloped by the occlusive braid, wherein the structural braid has a second pick angle;
wherein the structural braid is configured to drive the occlusive braid outward to press the occlusive braid against the tissue; and
the first pick angle of the occlusive braid is greater than the second pick angle of the structural braid; and
wherein the device has retention members integrated with the structural braid.

11. A device comprising:
an expandable lattice structure including—
an occlusive braid configured to contact and seal with tissue, wherein the occlusive braid has a first pick angle;
a structural braid enveloped by the occlusive braid, wherein the structural braid has a second pick angle;
wherein the structural braid is configured to drive the occlusive braid outward to press the occlusive braid against the tissue and the first pick angle of the occlusive braid is greater than the second pick angle of the structural braid; and
a generally cylindrical contact portion having—
a contact portion diameter;
a contact portion length measured along a longitudinal axis of the occlusion device;
wherein decreasing the contact portion diameter does not substantially change the length of the contact portion.

12. A device with an expandable lattice structure that comprises:
an occlusive braid configured to contact and seal with tissue, wherein the occlusive braid has a first pick angle;
a structural braid enveloped by the occlusive braid, wherein the structural braid has a second pick angle;
wherein the structural braid is configured to drive the occlusive braid outward to press the occlusive braid against the tissue, and the first pick angle of the occlusive braid is greater than the second pick angle of the structural braid; and
wherein the occlusive braid has a first pore sized and the structural braid has a second pore size that is greater than the first pore size.

13. A method for positioning a multilayered braided device, comprising:
positioning a multilayered braided device at anatomical site, the device having a structural braid and an occlusive braid around the structural braid;
expanding the structural braid such that the structural braid presses the occlusive braid against at least a portion of the anatomical site;
whereby the occlusive braid substantially seals to the anatomical site; and
wherein a first pick angle of the occlusive braid is greater than a second pick angle of the structural braid.

14. A method for positioning a multilayered braided device having a structural braid and an occlusive braid around the structural braid, comprising:
delivering the device in a contracted state with a elongate catheter;
positioning the catheter at anatomical site;
deploying the device at or near the anatomical;
expanding the structural braid such that the structural braid presses the occlusive braid against at least a portion of the anatomical site;
whereby the occlusive braid substantially seals to the anatomical site; and
wherein a first pick angle of the occlusive braid is greater than a second pick angle of the structural braid.
